(19) United States
(12) Patent Application Publication
(10) Pub. No.: US 2017/0080248 A1
(43) Pub. Date: Mar. 23, 2017

(54) METHOD AND EXTERNAL PREPARATION FOR LYMPHATIC SLIMMING

(71) Applicant: Satoshi SHINKAWA, Tokyo (JP)

(72) Inventor: Satoshi SHINKAWA, Tokyo (JP)

(21) Appl. No.: 15/369,120

(22) Filed: Dec. 5, 2016

Related U.S. Application Data
(62) Division of application No. 13/798,984, filed on Mar. 13, 2013.

(30) Foreign Application Priority Data
Sep. 27, 2012 (JP) 2012-213370

(51) Int. Cl. A61N 5/06; A61K 36/60; A61K 36/48; A61K 31/7048; A61K 36/232; A61K 36/185; A61K 31/575; A61K 31/355; A61N 5/10; A61K 36/062

(52) U.S. Cl. CPC A61N 5/062; A61N 5/1028; A61K 36/60; A61K 36/48; A61K 36/062; A61K 36/232; A61K 36/185; A61K 31/575; A61K 31/355; A61K 31/7048; A61N 2005/066; A61K 2236/19

(57) ABSTRACT

The present invention provides a set of body slimming compositions, for application to a body before irradiating the body with a far-infrared ray in an atmosphere of 50° C. or thereabout. The slimming composition set includes a first external composition containing a cosmetic base, a Bacillus-fig-soybean fermented liquor, a Bacillus-Monascus-angelica-soybean fermented liquor, a Bacillus-longan-soybean fermented liquor, orizanol, tocopheryl acetate, macadamia nut oil, and glucosyl hesperidin, and a second external composition containing a cosmetic base, a Bacillus-persimmon-soybean fermented liquor, a Waltheria indica leaf extract, and ascorbyl tetrahexyldecanoate. The first external composition is applied to a lymphatic node region of the body, and the second external composition is applied to a slimming target region of the body. The composition set of the invention can activate lymph or lipase, whereby a remarkable body slimming effect can be attained.

### BACKGROUND OF THE INVENTION

Field of the Invention

The present invention relates to a method for lymphatic slimming which includes irradiating a body, in particular a human body or a body part, with growth-promoting rays by means of slimming equipment while the body or body part is maintained at elevated temperature. The invention also relates to a set of external compositions for use in the lymphatic slimming method.

Background Art

One important function of the lymphatic system is to cause flow of lipid which has been taken into the lymphatic ducts from the tissue through contraction of skeletal muscle or the like, whereby the lipid is transported to the circulatory system via lymphatic nodes. As people get older, contraction of skeletal muscle or the like is weakened, and lipid accumulates in the lymphatic ducts, thereby causing obesity.

Several local body slimming techniques through removal of lipid accumulated in the lymphatic ducts have been performed, and examples of such techniques include local massage, acupuncture and moxibustion techniques, and surgical approaches. Also, a variety of external compositions for slimming have hitherto been proposed.

One type of such compositions is compositions which activate lipase. Specifically, Japanese Patent Application Laid-Open (kokai) No. 1999-116486 discloses a slimming composition containing cAMP as an active ingredient. Japanese Patent Application Laid-Open (kokai) No. 1999-269079 discloses a body slimming composition containing, as an active ingredient, an extract of a plant belonging to the Minispermaceae family. Japanese Patent Application Laid-Open (kokai) No. 1996-104618 and WO 2002/005755 disclose body slimming compositions containing as an active ingredient caffeine or a seaweed extract. Japanese Patent Publication (kokoku) No. 1991-68004 and Japanese Patent Application Laid-Open (kokai) No. 2000-63227 disclose body slimming compositions containing a plant extract as an active ingredient. Japanese Patent Application Laid-Open (kokai) No. 1995-228522 discloses a body slimming composition containing a herbal medicine as an active ingredient.

The present inventor previously proposed a local massage preparation containing ethoxyheptylbicyclooctanone, wild thyme extract, and licorice root extract. See Japanese Patent Application Laid-Open (kokai) Japanese Patent Application Laid-Open (kokai) No. 2007-15968.

Meanwhile, there has been known a body slimming method making use of a sauna bath apparatus disclosed in Japanese Patent Application Laid-Open (kokai) No. 2006-75320. The method includes irradiating a body with growth-promoting rays at about 40° C. for about one hour in the sauna bath.

The present inventor previously found a body slimming method capable of providing a slimming effect which cannot be obtained by simply applying an external composition containing a lipase-activating ingredient and a lymph-activating ingredient (Japanese Patent Application Laid-Open (kokai) No. 2011-1276). The method disclosed therein includes applying an external composition A previously developed by the inventor to a lymphatic node region of a body, applying an external composition B to a slimming target region of the body, and subsequently irradiating the slimming target region with growth-promoting rays, while the body is maintained in an atmosphere of 45° C. to 55° C., preferably at about 50° C., wherein the composition A comprises, as active ingredients, ethoxyheptylbicyclooctanone, wild thyme extract, and licorice root extract, and the composition B comprises ascorbyl tetrahexyldecanoate and a Waltheria indica leaf extract.

### SUMMARY OF THE INVENTION

The present inventor has conducted further studies, and have found that a remarkable body slimming effect can be attained by use, in combination, of a first external composition containing a fermented liquor produced from fig fruits, soybeans, and Bacillus subtilis natto, another fermented liquor produced from longan seeds, soybeans, and Bacillus subtilis natto, and yet another fermented liquor produced from Angelica keiskei leaves, soybeans, Bacillus subtilis natto, and Monasucus purpureus, and a second external composition containing a fermented liquor produced from persimmon leaves, soybeans, and Bacillus subtilis natto. Note that the first and second external compositions respectively correspond to “external composition A” and external composition B″ in the above paragraph.

In the present specification, for the sake of brevity and convenience, the fermented liquor produced from fig fruits, soybeans, and Bacillus subtilis natto is expressed as “Bacillus-fig-soybean fermented liquor,” the fermented liquor produced from longan seeds, soybeans, and Bacillus subtilis natto is expressed as “Bacillus-longan-soybean fermented liquor,” and a fermented liquor produced from Angelica keiskei leaves, soybeans, Bacillus subtilis natto, and Monasucus purpureus is expressed as “Bacillus-Monascus-angelica-soybean fermented liquor.”

The fermented liquor produced from persimmon leaves, soybeans, Bacillus subtilis natto is expressed as “Bacillus-persimmon-soybean fermented liquor.”

Accordingly, the present invention provides a set of external compositions for body slimming (hereinafter may be referred to as a “body slimming composition set”), for application to a body before a treatment of irradiating the body with a far-infrared ray for a predetermined period of time in an atmosphere of 50° C. or thereabout, comprising:

a first external composition containing a cosmetic base, a Bacillus-fig-soybean fermented liquor, a Bacillus-Monascus-angelica-soybean fermented liquor, a Bacillus-longan-soybean fermented liquor, orizanol, tocopheryl acetate, macadamia nut oil, and glucosyl hesperidin, and

a second external composition containing a cosmetic base, a Bacillus-persimmon-soybean fermented liquor, a Waltheria indica leaf extract, and ascorbyl tetrahexyldecanoate.

The present invention also provides a body slimming method, comprising:

applying, to a lymphatic node region of a body, a first external composition containing a cosmetic base, a Bacillus-fig-soybean fermented liquor, a Bacillus-Monascus-angelica-soybean fermented liquor, a Bacillus-longan-soybean fermented liquor, orizanol, tocopheryl acetate, macadamia nut oil, and glucosyl hesperidin;

applying, to a slimming target region of the body, a second external composition containing a cosmetic base, a Bacillus-persimmon-soybean fermented liquor, a Waltheria indica leaf extract, and ascorbyl tetrahexyldecanoate;

placing the body in slimming equipment; and

irradiating the body with a far-infrared ray for a predetermined period of time in an atmosphere of 50° C. or thereabout.

The novel features of the present invention will more fully appear from the following detailed description and the accompanying drawings.

### BRIEF DESCRIPTION OF THE DRAWINGS

FIG. 1 is a graph showing the treatment results in Example 1; and

FIG. 2 is a graph showing the treatment results in Example 2.

### DETAILED DESCRIPTION OF PREFERRED EMBODIMENTS

The body slimming composition set of the present invention, for application to a body before irradiating the body with a far-infrared ray for a predetermined period of time in an atmosphere of 50° C. or thereabout, comprises:

a first external composition containing a cosmetic base, a Bacillus-fig-soybean fermented liquor, a Bacillus-Monascus-angelica-soybean fermented liquor, a Bacillus-longan-soybean fermented liquor, orizanol, tocopheryl acetate, macadamia nut oil, and glucosyl hesperidin, and

a second external composition containing a cosmetic base, a Bacillus-persimmon-soybean fermented liquor, a Waltheria indica leaf extract, and ascorbyl tetrahexyldecanoate.

The Bacillus-fig-soybean fermented liquor may be produced through a method well known in the art by fermenting fig fruits and soybeans in the presence of Bacillus subtilis natto. The other fermented liquors employed in the first and second external compositions may also be produced in a similar manner. The fermented liquors may be in the form of extract.

In one method of producing the Bacillus-fig-soybean fermented liquor, Bacillus subtilis natto is added to dried powder of fig fruits and soybean powder, and the mixture is allowed to ferment at 30° C. to 45° C. for 2 days to 14 days.

In one method of producing the Bacillus-longan-soybean fermented liquor, Bacillus subtilis natto is added to longan seed powder and soybean powder, and the mixture is allowed to ferment at 30° C. to 55° C. for 28 hours to 72 hours.

In one method of producing the Bacillus-Monascus-angelica-soybean, Bacillus subtilis natto is added to powder of dried Angelica keiskei leaves and soybean powder, and the mixture is allowed to ferment at 30° C. to 45° C. for 2 days. Subsequently, Monasucus purpureus is added to the fermentation product, and the resultant mixture is allowed to ferment at 37° C. for 2 to 7 days.

In one method of producing the Bacillus-persimmon-soybean fermented liquor, Bacillus subtilis natto is added to powder of dry persimmon leaves and soybean powder, and the mixture is allowed to ferment at 30 to 45° C. for 2 to 14 days.

In the above production methods, all fermentation processes are carried out aerobically.

The first external composition of the present invention is applied to the skin or one or more body parts (regions) corresponding to major lymphatic nodes for promoting the flow of lymphatic fluid.

Through promotion of lymphatic flow, cell proliferation and metabolism are facilitated, thereby enhancing permeation of body fluid to cells. When active ingredients permeate cells, anti-oxidation and fat decomposition are further promoted, whereby waste matter can be excreted, and circulation of lymph and blood can be promoted. The Bacillus-fig-soybean fermented liquor of the present invention, having a very high cell proliferation effect, is thought to enhance the metabolism-promoting effect.

The Bacillus-Monascus-angelica-soybean fermented liquor of the present invention is thought to enhance fat decomposition effect.

Meanwhile, collagen activates metabolism, feeds oxygen and nutrients to cells, smoothly excretes waste matter from the body, enhances fat burning efficiency, and suppresses accumulation of fat. The Bacillus-longan-soybean fermented liquor of the present invention enhances regeneration of collagen.

Tocopheryl acetate, macadamia nut oil, and glucosyl hesperidin promote circulation of lymph and blood. Orizanol promotes excretion of waste matter.

The second external composition is applied to a slimming target region. The Bacillus-persimmon-soybean fermented liquor contained in the second external composition increases the collagen level at the application site, to thereby enhance fat burning effect and suppress fat accumulation.

The Waltheria indica leaf extract exhibits various effects such as cell rejuvenation, and enhancement of metabolism and permeation ability.

Ascorbyl tetrahexyldecanoate prevents oxidation.

The second external composition of the present invention contains ascorbyl tetrahexyldecanoate and a Waltheria indica leaf extract as active ingredients.

Of these ingredients, ascorbyl tetrahexyldecanoate decomposes and eliminates active oxygen, which triggers generation of fat waste, whereby a slimming effect is obtained.

Waltheria indica is a herb belonging to the group Briza minor and is found in French Guiana, South America. The extract thereof has a whitening action and an active oxygen removal action. When the extract is used in combination with ascorbyl tetrahexyldecanoate and an additional anti-bacterial ingredient such as ferulic acid, a remarkably excellent body slimming effect can be attained.

The first and second external compositions of the present invention contain a cosmetic base (basic ingredient), which is known per se in this technical field.

The growth-promoting rays employed in the present invention are far infrared rays having a wavelength of 6 to 14 μm. When the body is irradiated with the growth-promoting rays, motions of water molecules present in the body are activated, resulting in dilation of capillary vessels, promotion of blood circulation, activation of the lymphatic system and lipase, promotion of metabolism, etc. The growth-promoting rays may be obtained by any far infrared sources (lamps) known in the art.

Since the irradiation is performed in an atmosphere of 45° C. to 55° C. (preferably about 50° C.), the aforementioned dilation of capillary vessels, promotion of blood circulation, activation of the lymphatic system and lipase, promotion of metabolism, etc. can more smoothly proceed.

Therefore, according to the present invention, the body slimming effect can be attained simply through applying the first and second external compositions to a body and irradiating the body with growth-promoting rays for about one hour.

A higher body slimming effect may be attained in the following manner. Specifically, the first and second external compositions are applied to a body, and the body is wrapped with a heat-insulating covering, such as a blanket or a mat, whose temperature is controlled at about 50° C. and maintained for about one hour. The heat-insulating covering has a substrate to which a radioactive element mineral radiating a growth-promoting ray, such as a radium-containing mineral or a thorium-containing mineral, has been incorporated, and a temperature-controllable heater disposed on the substrate. Then, the body is given such a massage for transferring lymph to the lymphatic nodes.

Thus, according to the present invention, specific external compositions, each containing ingredients which can activate lymph or lipase, are applied to a body, and the body is irradiated with growth-promoting rays at a specific temperature for a specific period of time, optionally in combination with use of temperature-controllable heat-insulating equipment and massage, whereby a remarkable body slimming effect can be attained.

### EXAMPLES

The body slimming first and second external compositions and the body sliming method of the present invention will be described in detail by way of examples, which should not be construed as limiting the invention thereto.

### Example 1

(1) First external composition:

| | | |
|---|---|---|
| a | Cosmetic base | 96.2 wt. % |
| b | Bacillus-fig-soybean fermented liquor | 1.0 wt. % |
| c | Bacillus-Monascus-angelica-soybean | 0.8 wt. % |
| | fermented liquor | |
| d | Bacillus-longan-soybean fermented liquor | 0.8 wt. % |
| e | Orizanol | 0.3 wt. % |
| f | Tocopheryl acetate | 0.3 wt. % |
| g | Macadamia nut oil | 0.3 wt. % |
| h | Glucosyl hesperidin | 0.3 wt. % |

The first external composition was prepared by mixing all the ingredients at about 25° C. (room temperature), and then allowing the mixture to stand for 4 days.

(2) Second external composition

| | | |
|---|---|---|
| a | Cosmetic base | 97.2 wt. % |
| b | Bacillus-persimmon-soybean fermented liquor | 1.0 wt. % |
| c | Waltheria indica leaf extract | 0.9 wt. % |
| d | Ascorbyl tetrahexyldecanoate | 0.9 wt. % |

The second external composition was prepared by mixing all the ingredients at about 25° C. (room temperature), and then allowing the mixture to stand for 4 days.

### (3) Treatment Example

A slimming treatment of Example 1 was applied to 30 subjects. Specifically, the first external composition was applied to the skin of main lymphatic node regions of a body of each subject, and the second external composition was applied to the abdomen skin of the body. The main lymphatic node regions to which the first external composition was applied included a supraclavicular lymphatic node region, a parotid lymphatic node region, an axillary lymphatic node region, an inguinal lymphatic node region, and a popliteal lymphatic node region. The subject was wrapped with a heat-insulating covering and maintained at about 50° C. for about one hour. The heat-insulating covering had a substrate containing a radioactive element mineral for radiating a growth-promoting ray (a mixture containing a radium-containing mineral and a thorium-containing mineral) and a temperature-controllable heater disposed on the substrate.

### (4) Treatment Results

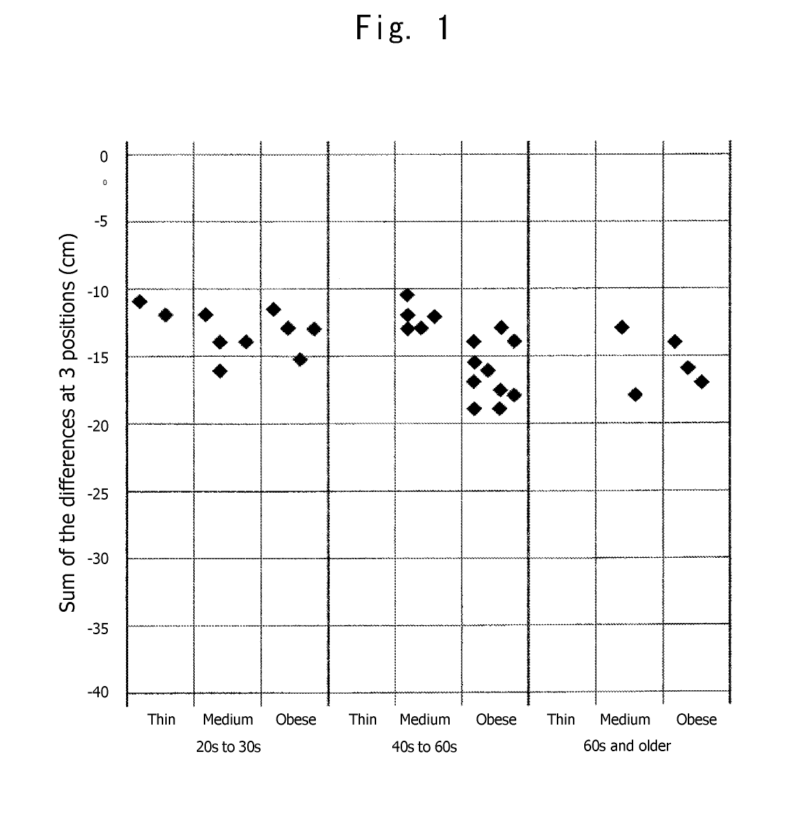

Each of the 30 subjects in their twenties to seventies who were thin, medium, or obese received the above treatment. Before and after the treatment, the waist circumference of the subject was measured at a position 5 cm above the navel, at the navel, and at a position 5 cm below the navel. In each case, the difference between before and after was calculated. Table 1 shows the measured differences and the sums of three differences. FIG. 1 shows the results.

TABLE 1

| | | | Above navel | | | Navel | | | Below navel | | | Sum of |
|---|---|---|---|---|---|---|---|---|---|---|---|---|
| | Age | Body type | Before | After | Δ | Before | After | Δ | Before | After | Δ | Δ |
| 1 | 35 | Thin | 70.00 | 66.00 | 4.00 | 78.00 | 74.00 | 4.00 | 87.00 | 84.00 | 3.00 | 11.00 |
| 2 | 24 | Medium | 75.00 | 71.50 | 3.50 | 83.00 | 78.50 | 4.50 | 88.00 | 84.00 | 4.00 | 12.00 |
| 3 | 40 | Obese | 92.00 | 86.20 | 5.80 | 92.50 | 87.40 | 5.10 | 97.50 | 94.40 | 3.10 | 14.00 |
| 4 | 54 | Medium | 78.80 | 72.70 | 6.10 | 88.00 | 83.60 | 4.40 | 98.60 | 96.10 | 2.50 | 13.00 |
| 5 | 48 | Medium | 74.00 | 69.80 | 4.20 | 78.50 | 74.20 | 4.30 | 82.00 | 80.00 | 2.00 | 10.50 |
| 6 | 36 | Medium | 80.00 | 75.50 | 4.50 | 87.00 | 80.00 | 7.00 | 90.00 | 85.50 | 4.50 | 16.00 |
| 7 | 29 | Medium | 79.00 | 74.40 | 4.60 | 85.00 | 79.00 | 6.00 | 94.50 | 91.10 | 3.40 | 14.00 |
| 8 | 39 | Obese | 105.50 | 100.50 | 5.00 | 109.50 | 105.80 | 3.70 | 109.50 | 106.70 | 2.80 | 11.50 |
| 9 | 40 | Medium | 79.50 | 73.70 | 5.80 | 89.50 | 85.20 | 4.30 | 93.50 | 91.60 | 1.90 | 12.00 |
| 10 | 25 | Thin | 70.00 | 66.00 | 4.00 | 79.00 | 74.00 | 5.00 | 85.00 | 82.00 | 3.00 | 12.00 |
| 11 | 45 | Medium | 75.00 | 70.80 | 4.20 | 79.00 | 74.20 | 4.80 | 80.00 | 77.00 | 3.00 | 12.00 |
| 12 | 63 | Medium | 74.50 | 68.80 | 5.70 | 82.50 | 78.20 | 4.30 | 86.00 | 83.00 | 3.00 | 13.00 |
| 13 | 52 | Obese | 89.00 | 81.60 | 7.40 | 92.00 | 87.30 | 4.70 | 96.00 | 92.60 | 3.40 | 15.50 |
| 14 | 20 | Obese | 102.00 | 95.50 | 6.50 | 98.00 | 94.00 | 4.00 | 103.50 | 101.00 | 2.50 | 13.00 |
| 15 | 45 | Obese | 93.00 | 87.00 | 6.00 | 94.00 | 87.80 | 6.20 | 96.00 | 94.20 | 1.80 | 14.00 |
| 16 | 55 | Obese | 81.50 | 74.00 | 7.50 | 88.00 | 82.00 | 6.00 | 91.50 | 88.00 | 3.50 | 17.00 |
| 17 | 62 | Obese | 99.00 | 94.30 | 4.70 | 104.00 | 99.00 | 5.00 | 109.50 | 105.20 | 4.30 | 14.00 |
| 18 | 39 | Obese | 93.00 | 87.30 | 5.70 | 99.00 | 94.60 | 4.40 | 101.00 | 95.90 | 5.10 | 15.20 |
| 19 | 56 | Obese | 92.00 | 82.50 | 9.50 | 95.00 | 89.00 | 6.00 | 95.00 | 91.50 | 3.50 | 19.00 |
| 20 | 45 | Obese | 100.00 | 93.70 | 6.30 | 107.00 | 100.00 | 7.00 | 105.00 | 102.30 | 2.70 | 16.00 |
| 21 | 54 | Obese | 87.00 | 80.50 | 6.50 | 93.50 | 86.20 | 7.30 | 98.50 | 94.30 | 4.20 | 18.00 |
| 22 | 76 | Obese | 88.00 | 81.10 | 6.90 | 94.00 | 88.00 | 6.00 | 98.00 | 94.90 | 3.10 | 16.00 |
| 23 | 40 | Obese | 84.80 | 78.80 | 6.00 | 88.90 | 83.80 | 5.10 | 91.80 | 85.40 | 6.40 | 17.50 |
| 24 | 45 | Obese | 86.20 | 76.80 | 9.40 | 90.00 | 85.70 | 4.30 | 96.00 | 90.70 | 5.30 | 19.00 |
| 25 | 35 | Obese | 96.00 | 91.00 | 5.00 | 106.00 | 100.30 | 5.70 | 106.00 | 103.70 | 2.30 | 13.00 |
| 26 | 64 | Medium | 78.00 | 71.00 | 7.00 | 89.50 | 82.90 | 6.60 | 95.00 | 90.60 | 4.40 | 18.00 |
| 27 | 39 | Medium | 79.00 | 72.40 | 6.60 | 81.00 | 76.00 | 5.00 | 85.00 | 82.60 | 2.40 | 14.00 |
| 28 | 40 | Medium | 73.20 | 66.60 | 6.60 | 88.60 | 84.30 | 4.30 | 94.50 | 92.40 | 2.10 | 13.00 |
| 29 | 42 | Obese | 87.00 | 81.70 | 5.30 | 95.00 | 90.80 | 4.20 | 98.00 | 94.50 | 3.50 | 13.00 |
| 30 | 71 | Obese | 95.00 | 86.00 | 9.00 | 105.00 | 100.00 | 5.00 | 108.00 | 105.00 | 3.00 | 17.00 |

### Example 2

### (1) Treatment Example

A body slimming treatment of Example 2 was applied to another 30 subjects. Specifically, each subject received the treatment of Example 1 (3) and, subsequently, a massage for transferring lymph to the lymphatic nodes was applied to the subject (i.e., combination treatment).

### (2) Effects of the Treatment

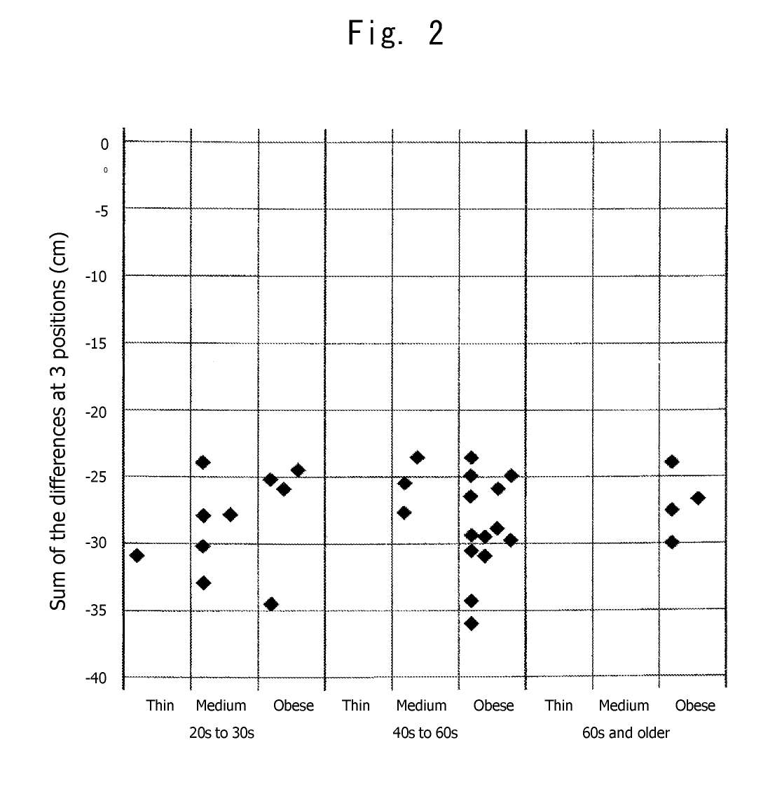

Each of the 30 subjects in their twenties to seventies who were thin, medium, or obese received the treatment of Example 2. Before and after the treatment, the waist circumference of the subject was measured at a position 5 cm above the navel, at the navel, and at a position 5 cm below the navel. In each case, the difference between before and after was calculated. Table 2 shows the measured differences and the sums of three differences. FIG. 2 shows the results.

TABLE 2

| | | | Above navel | | | Navel | | | Below navel | | | Sum of |
|---|---|---|---|---|---|---|---|---|---|---|---|---|
| | Age | Body type | Before | After | Δ | Before | After | Δ | Before | After | Δ | Δ |
| 1 | 54 | Obese | 112.00 | 101.00 | 11.00 | 116.00 | 101.00 | 15.00 | 114.00 | 104.00 | 10.00 | 36.00 |
| 2 | 24 | Medium | 75.00 | 65.50 | 9.50 | 83.00 | 73.00 | 10.00 | 88.00 | 79.50 | 8.50 | 28.00 |
| 3 | 40 | Obese | 92.00 | 81.25 | 10.75 | 92.50 | 84.50 | 8.00 | 97.50 | 87.00 | 10.50 | 29.25 |
| 4 | 54 | Obese | 108.00 | 99.50 | 8.50 | 108.00 | 100.25 | 7.75 | 114.00 | 103.75 | 10.25 | 26.50 |
| 5 | 48 | Medium | 74.00 | 67.00 | 7.00 | 78.50 | 70.00 | 8.50 | 82.00 | 72.00 | 10.00 | 25.50 |
| 6 | 36 | Medium | 80.00 | 70.00 | 10.00 | 87.00 | 75.20 | 11.80 | 90.00 | 81.50 | 8.50 | 30.30 |
| 7 | 29 | Medium | 79.00 | 69.00 | 10.00 | 85.00 | 73.00 | 12.00 | 94.50 | 83.50 | 11.00 | 33.00 |
| 8 | 39 | Obese | 105.50 | 96.20 | 9.30 | 109.50 | 100.00 | 9.50 | 109.50 | 103.00 | 6.50 | 25.30 |
| 9 | 40 | Medium | 79.50 | 70.00 | 9.50 | 89.50 | 79.00 | 10.50 | 93.50 | 85.70 | 7.80 | 27.80 |
| 10 | 32 | Thin | 70.00 | 60.00 | 10.00 | 79.00 | 67.00 | 12.00 | 85.00 | 76.00 | 9.00 | 31.00 |
| 11 | 45 | Obese | 85.00 | 72.50 | 12.50 | 89.00 | 76.70 | 12.30 | 90.00 | 80.50 | 9.50 | 34.30 |
| 12 | 63 | Obese | 84.50 | 75.50 | 9.00 | 92.50 | 83.50 | 9.00 | 96.00 | 86.50 | 9.50 | 27.50 |
| 13 | 52 | Obese | 89.00 | 77.50 | 11.50 | 92.00 | 82.00 | 10.00 | 96.00 | 87.00 | 9.00 | 30.50 |
| 14 | 20 | Obese | 102.00 | 88.50 | 13.50 | 98.00 | 88.00 | 10.00 | 103.50 | 92.50 | 11.00 | 34.50 |
| 15 | 45 | Obese | 93.00 | 85.00 | 8.00 | 94.00 | 80.00 | 14.00 | 96.00 | 87.00 | 9.00 | 31.00 |
| 16 | 55 | Obese | 81.50 | 72.00 | 9.50 | 88.00 | 80.00 | 8.00 | 91.50 | 85.50 | 6.00 | 23.50 |
| 17 | 62 | Obese | 99.00 | 89.00 | 10.00 | 104.00 | 94.00 | 10.00 | 109.50 | 99.50 | 10.00 | 30.00 |
| 18 | 39 | Obese | 93.00 | 84.00 | 9.00 | 99.00 | 89.00 | 10.00 | 101.00 | 94.00 | 7.00 | 26.00 |
| 19 | 56 | Obese | 92.00 | 80.50 | 11.50 | 95.00 | 84.00 | 11.00 | 95.00 | 88.00 | 7.00 | 29.50 |
| 20 | 45 | Obese | 100.00 | 90.00 | 10.00 | 107.00 | 98.00 | 9.00 | 105.00 | 99.00 | 6.00 | 25.00 |
| 21 | 54 | Obese | 87.00 | 77.00 | 10.00 | 93.50 | 82.00 | 11.50 | 98.50 | 91.00 | 7.50 | 29.00 |
| 22 | 76 | Obese | 88.00 | 78.00 | 10.00 | 94.50 | 88.00 | 6.00 | 98.00 | 90.00 | 8.00 | 24.00 |
| 23 | 40 | Obese | 84.80 | 72.80 | 12.00 | 88.90 | 78.80 | 10.10 | 91.80 | 84.60 | 7.20 | 29.30 |
| 24 | 45 | Obese | 86.20 | 76.70 | 9.50 | 90.00 | 80.80 | 9.20 | 96.00 | 88.70 | 7.30 | 26.00 |
| 25 | 35 | Obese | 96.00 | 87.50 | 8.50 | 106.00 | 97.00 | 9.00 | 106.00 | 99.00 | 7.00 | 24.50 |
| 26 | 44 | Medium | 78.00 | 70.50 | 7.50 | 89.50 | 80.30 | 9.20 | 95.00 | 88.00 | 7.00 | 23.70 |
| 27 | 39 | Medium | 79.00 | 70.00 | 9.00 | 81.00 | 74.00 | 7.00 | 85.00 | 77.00 | 8.00 | 24.00 |
| 28 | 30 | Medium | 73.20 | 66.10 | 7.10 | 88.60 | 77.00 | 11.60 | 94.50 | 85.20 | 9.30 | 28.00 |
| 29 | 62 | Obese | 87.00 | 78.80 | 8.20 | 95.00 | 84.50 | 10.50 | 98.00 | 90.00 | 8.00 | 26.70 |
| 30 | 41 | Obese | 95.00 | 86.00 | 9.00 | 105.00 | 95.00 | 10.00 | 108.00 | 102.00 | 6.00 | 25.00 |

According to the present invention, specific external compositions, each containing ingredients which can activate lymph or lipase, are applied to a body, and the body is irradiated with growth-promoting rays at a specific temperature for a specific period of time, whereby a remarkable body slimming effect can be attained.

1. (canceled)

2. A body slimming method, comprising
applying, to a lymphatic node region of a body, a first external composition containing a cosmetic base, a Bacillus-fig-soybean fermented liquor, a Bacillus-Monascus-angelica-soybean fermented liquor, a Bacillus-longan-soybean fermented liquor, orizanol, tocopheryl acetate, macadamia nut oil, and glucosyl hesperidin;
applying, to a slimming target region of the body, a second external composition containing a cosmetic base, a Bacillus-persimmon-soybean fermented liquor, a Waltheria indica leaf extract, and ascorbyl tetrahexyldecanoate;
placing the body in slimming equipment; and
irradiating the body with a far-infrared ray for a predetermined period of time in an atmosphere of 50° C. or thereabout.

3. A method according to claim 2, wherein the slimming equipment is a heat-insulating covering having a substrate to which a radioactive element mineral radiating a growth-promoting ray has been incorporated, and a temperature-controllable heater disposed on the substrate.
